(19) United States
(12) Patent Application Publication
(10) Pub. No.: US 2018/0344350 A1
(43) Pub. Date: Dec. 6, 2018

(54) Instrument System For Minimally Invasive Surgery In A Patient's Tissue

(71) Applicants: Thomas Gaiselmann, Villingendorf (DE); Siegfried Riek, Rottweil (DE); Karl-Heinz Bachmann, Villingendorf (DE)

(72) Inventors: Thomas Gaiselmann, Villingendorf (DE); Siegfried Riek, Rottweil (DE); Karl-Heinz Bachmann, Villingendorf (DE)

(21) Appl. No.: 15/991,720

(22) Filed: May 29, 2018

(30) Foreign Application Priority Data
May 30, 2017 (DE) 10 2017 111 821.7

(51) Int. Cl. A61B 17/34; A61B 17/00

(52) U.S. Cl. CPC A61B 17/3421; A61B 17/320016; A61B 17/00234; A61B 17/3498

(57) ABSTRACT

An instrument system for minimally invasive surgery in the tissue of a patient, with an instrument shaft, having a distal end that can be inserted in the body of the patient and a proximal end that remains outside the body, with an inner channel extending axially and continuously in the instrument shaft, with at least one working channel extending axially and continuously in the instrument shaft, and with an obturator which can accommodate an optical system coaxially and can be inserted into the inner channel in such a manner that its transparent distal tip projects out from the beveled distal front end of the instrument shaft. The system can include a valve block at the proximal end of the instrument shaft, closing the inner channel and enabling sealed passage of the obturator or an optical system.

### CROSS-REFERENCE TO RELATED APPLICATIONS

This application claims priority to German Patent Application No. 10 2017 111 821.7, filed May 30, 2017, which is incorporated by reference in its entirety.

### BACKGROUND

The present application relates to an instrument system for minimally invasive surgery in the tissue of a patient.

### SUMMARY

Minimally invasive surgery in natural body cavities, for example in the abdominal cavity, is performed using trocars, which consist of a trocar obturator and a trocar sleeve. The trocar obturator is used to pierce the tissue surrounding the body cavity in order to insert the trocar sleeve. Gas is insufflated into the body cavity to expand the cavity and create an adequate surgical field. The obturator is then withdrawn and the trocar sleeve remains, to provide access to the surgical field for an endoscopic optical system and for surgical instruments.

In the case of minimally invasive surgery in the tissue of a patient, for example, surgical removal of a tumor embedded in the tissue, a problem exists in that no natural cavity is present for the surgical procedure. Therefore, US 2015/0051495 A1 describes a method for minimally invasive surgery in the tissue of a patient, e.g., for removing a tumor in the female breast, in which a trocar with an optical obturator is made to pierce into the tissue under visual observation until the distal tip of the trocar reaches the tumor to be dissected. Then pressurized gas is insufflated through the trocar to force the tissue apart and create an artificial cavity for the surgery to be performed. Instruments can be introduced through the working channel of the trocar sleeve to perform the steps of the surgery in front of the distal tip of the trocar.

A trocar with an obturator, a trocar sleeve and an endoscopic optical system is known from DE 10 2011 107 615 A1.

The present disclosure is based on the task of providing an instrument system for minimally invasive surgery in the tissue of a patient, which allows the dissection of an artificial surgical cavity for the surgical procedure.

According to the present disclosure, this task is accomplished using an instrument system having the features and structures recited herein.

Advantageous embodiments of the present disclosure are further recited herein.

The instrument system according to the present disclosure has an elongated instrument shaft of stable form, which is introduced into the body of the patient. The distal end of the instrument shaft then enters intracorporeally into the target area of the tissue, while the proximal end of the instrument shaft remains outside of the body. The instrument shaft has an inner channel extending axially in a continuous, straight line from the proximal to the distal end. Into this inner channel, an obturator can be inserted, which has a distal transparent tip and can accommodate an optical system by which during the penetration of the tissue, the tissue adjacent to the transparent tip can be visualized to observe the penetration and positioning of the distal tip. Furthermore the instrument shaft has at least one working channel, which is likewise formed axially, extending continuously from the proximal to the distal end. Semi-flexible instruments can be introduced through the working channels in such a manner that their distal working elements emerge distally from the working channel and can be used in front of the distal tip of the instrument shaft, while the operating elements disposed at the proximal end of the instrument remain outside of the body. Furthermore, a pressurized gas can be insufflated through a working channel to create an artificial cavity for the surgical operation to be performed. In addition, if necessary, irrigation fluid can be introduced through a working channel and/or blood and tissue fluid can be suctioned off.

At the proximal end of the inner channel, a valve block is disposed on the instrument shaft. The valve block provides sealed closure for the inner channel when no instrument is introduced into the inner channel. When the obturator is introduced into the inner channel, the valve block seals the obturator in the inner channel at its outer circumference. When the obturator is withdrawn after positioning of the distal tip in the channel, in place of the obturator an endoscopic optical system can be introduced into the inner channel so that the surgical procedure can be performed under direct view. In this case the endoscopic optical system is also sealed off by the valve block in the inner channel.

The inner channel follows a straight line in the instrument shaft up to its proximal end, so that the essentially rigid straight obturator and the optics can be introduced into this inner channel. On the other hand, at their proximal end region, which remains outside of the body during surgery, the working channels are bent or curved away from the axis of the instrument shaft toward the outside, resulting in an ergonomically advantageous position for operating the instruments inserted in the working channel.

Such a bent shape of the working channels is possible through the use of semi-flexible instruments. At the proximal ends of the working channels as well, valves are disposed that allow the instruments to be introduced while maintaining a tight seal and allow the working channels to be closed with a seal when no instruments are introduced in the working channel. These valves also allow for the insufflation of the pressurized gas.

Furthermore, at the proximal end of the instrument shaft, two hand grips are preferably molded on, which facilitate the controlled advancement and movement of the instrument shaft by the surgeon. It is advantageous if the proximal ends of the working channels are spatially disposed relative to one another so that operator, using only one hand, can hold a hand grip and at the same time operate an instrument disposed in the corresponding working channel.

The distal end of the instrument shaft is beveled such that the plane of the distal front end of the instrument shaft deviates from the axis of the instrument shaft at an angle different from 90°.

When the obturator is inserted in the instrument shaft, its penetrating distal tip projects beyond the distal end face of the instrument shaft to allow penetration of the tissue. Here, the distal end of the obturator is seated with its external circumference closely fitted in the internal diameter of the inner channel to prevent penetration of tissue and the like into the inner channel when the distal tip is being advanced.

In this process, the end face of the instrument shaft preferably passes continuously into the circumferential curved surface area of the tip of the obturator, so that the tissue penetrated by the tip of the obturator can undergo further blunt dissection by the subsequent distal end of the instrument shaft.

The axial position of the obturator in the instrument shaft can be accurately maintained by suitable fixing aids. As soon as the distal tip is positioned in the surgical target area, the obturator is withdrawn. The optical system is introduced into the inner channel until its distal end is positioned at the distal outlet of the inner channel. Thus the optical system allows direct view into the surgical field. The optical system can also be fixed in its position in the instrument shaft with an optical system clamp. Since penetration of the tissue is no longer occurring in this stage, the distal end of the optical system need not be seated with a tight seal in the inner channel. Therefore it is possible to use the same endoscopic optical system inserted in the obturator for observing the surgical field even without the obturator. To prevent penetration of tissue, fluid and the like into the distally open working channels during penetration, the working channels may also be closed by an inserted stylet if desired.

During penetration into the tissue, the tissue is penetrated by the distally projecting tip of the obturator, while the beveled distal end of the instrument shaft performs blunt dissection of the penetrated tissue. As soon as the distal tip has reached the surgical target area, the instrument shaft can be rotated around its longitudinal axis, as a result of which, because of the beveled tip of the instrument shaft and the eccentric arrangement of the obturator tip, an annular cavity is dissected in the tissue, enabling the insufflation of pressurized gas.

The instrument shaft is preferably manufactured as a one-piece plastic article.

### BRIEF DESCRIPTION OF THE DRAWINGS

In the following, the present application will be explained in greater detail based on an embodiment shown in the drawings. The following are shown:

FIG. 1 a perspective view of an instrument system according to the present disclosure,

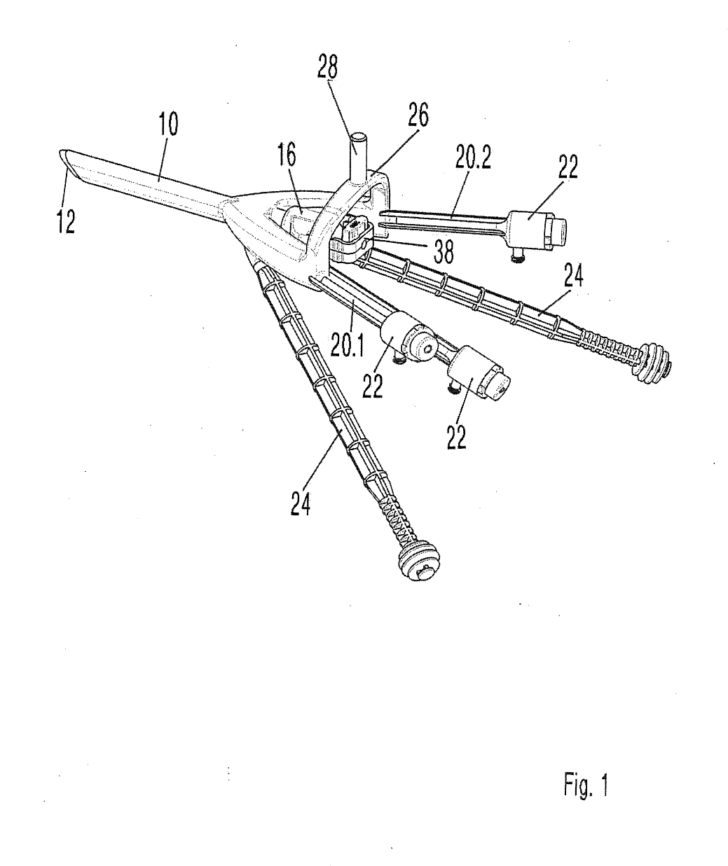

FIG. 2 a side view of the instrument system,

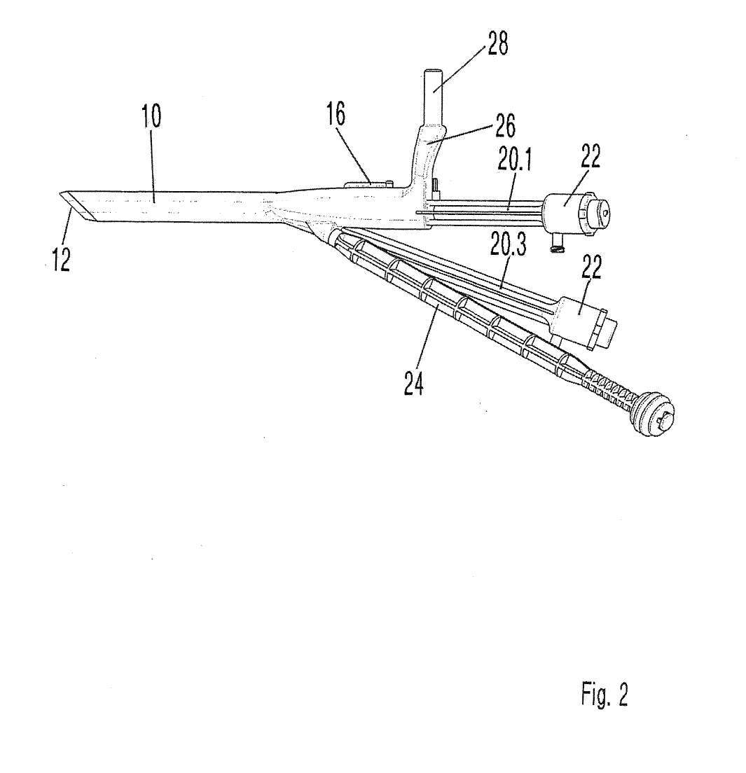

FIG. 3 a plan view of the instrument system from the top,

FIG. 4 an axial view of the proximal end of the instrument system,

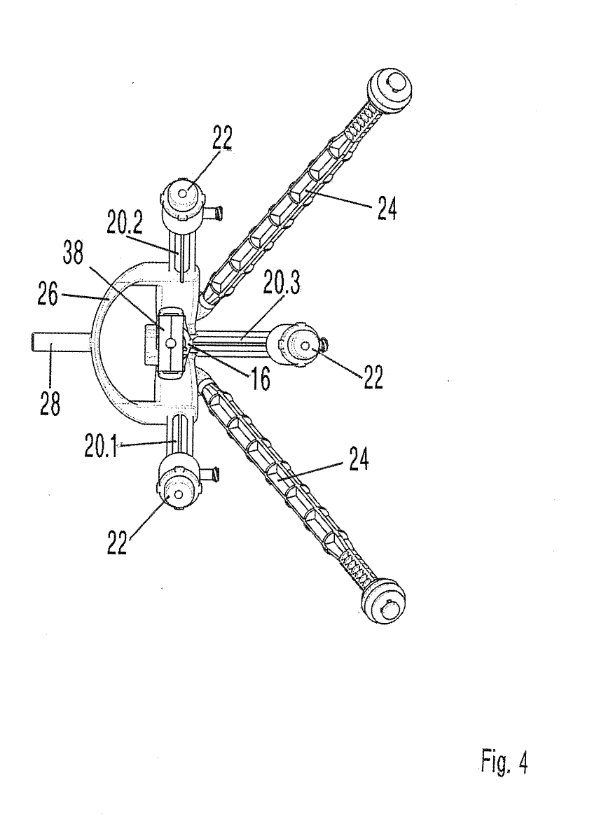

FIG. 5 an axial view of the distal end of the instrument system,

FIG. 6 the instrument system with an obturator,

FIG. 7 a plan view corresponding to FIG. 3 of the instrument system with the obturator inserted,

FIG. 8 an axial view of the distal front face of the instrument shaft and

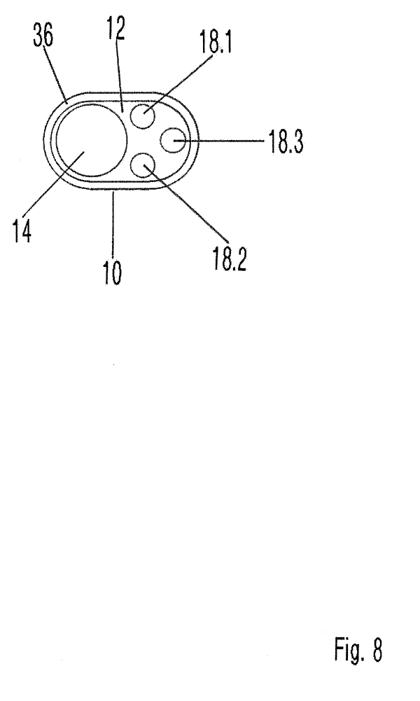

FIG. 9 an axial section of the distal end of the instrument system with obturator inserted.

### DETAILED DESCRIPTION

In the embodiments shown, the instrument system has an instrument shaft 10, which is designed as an elongated, straight cylinder with an approximately oval cross-section. The distal anterior face 12 of the instrument shaft 10 is beveled such that its plane forms an angle differing from 90° with the axis of the instrument shaft 10; this is preferably between 30° and 60° and in the embodiment shown is 45°.

Through the instrument shaft 10 in the axial longitudinal direction thereof, an inner channel 14 passes, which preferably has a circular cross-section and proceeds in a continuous straight line from the proximal end to the distal end of the instrument shaft 10. The inner channel 14 proceeds in the cross-section of the oval instrument shaft 10, preferably eccentric to the middle axis of the instrument shaft 10. As a result, the inner channel 14 at the distal end emerges in the further distally located surface of the beveled end face. A valve block 16 is located at the proximal end of the inner channel 14. The valve block 16 preferably has an internal valve flap and a lip seal. The valve flap closes the inner channel 14 airtight and can be opened to permit the passage of an instrument. When an instrument is introduced into the inner channel 14 and the valve flap is opened, the lip seal abuts against the circumference of the instrument introduced and thus closes off the inner channel 14.

Furthermore, at least one working channel is formed in the instrument shaft 10. In the embodiment shown, three working channels 18 are provided.

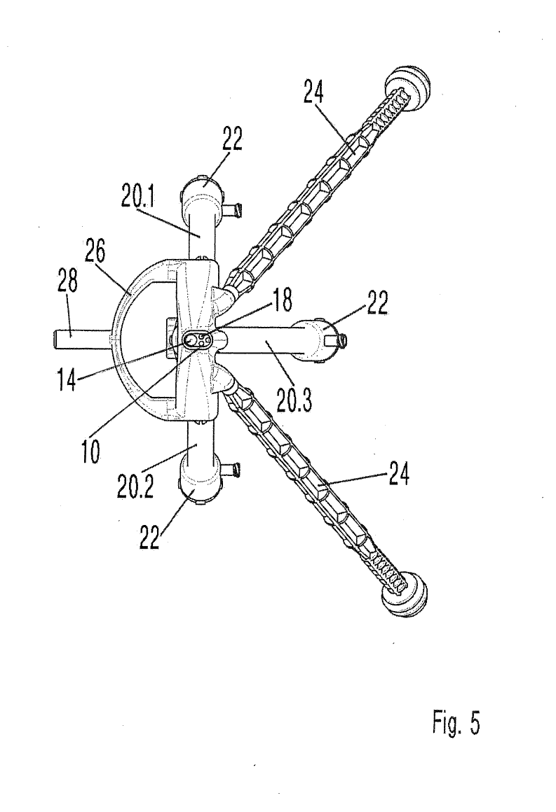

The working channels 18 travel in a direction parallel to the axis alongside the inner channel 14 in the instrument shaft 10 and open distally in the end face 12 in the surface area thereof, located further proximally. As is shown in FIG. 5, the three working channels 18 are disposed in a triangle in the cross-section of the instrument shaft 10 beneath the inner channel 14.

The working channels 18 travel in a straight line parallel to the axis in the instrument shaft 10 to the proximal end of the instrument shaft 10. At the proximal end of the instrument shaft 10, the working channels 18 proceed toward the longitudinal access of the instrument shaft 10 bent toward the outside, so that with the axis of the instrument shaft 10 they form a proximally opening angle preferably between 15° and 45°, and in the embodiment shown about 30°. The angled proximal end region of the working channels 18 in each case is formed by a tubular appendage 20, in each case formed in one piece on the proximal end of the instrument shaft 10. The tubular appendages 20 that enclose the working channel 18 extend in the proximal direction beyond the proximal end of the instrument shaft 10 and the valve block 16. In the embodiment shown, two appendages 20.1 and 20.2 with the respective working channels 18.1 and 18.2 are disposed in a plane that encloses the axis of the instrument shaft 10. The third appendage 20.3 with the working channel 18.3 is bent downward from this plane.

At the proximal end of the appendage 20 in each case a valve 22 is disposed, which seals off the respective end of the working channel 18 and allows sealed introduction of an instrument as is explained above for the valve block 16.

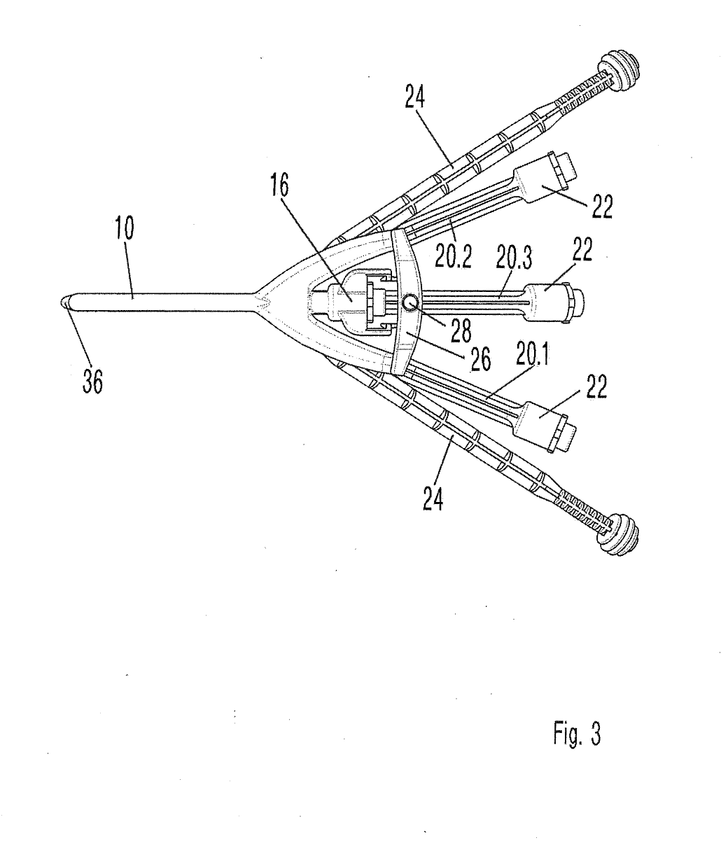

In addition at least one hand grip 24 is integrally formed at the proximal end of the instrument shaft 10. In the embodiments shown, two hand grips 24 are present. The hand grips 24 are designed as integrally formed rods that are angled against the axis of the instrument shaft 10 and form with the axis of the instrument shaft 10 a proximally opening angle of about 15° to 45°, preferably of about 30°. As is especially recognizable from FIG. 3, the two hand grips 24 are essentially below the appendages 20.1 and 20.2.

At the proximal end of the instrument shaft 10, a bracket is also integrally formed, preferably extending in an arc shape from the appendages 20.1 and 20.2. On the bracket 26 a connector 28 is integrally formed for fastening the instrument shaft 10 to a stand or the like.

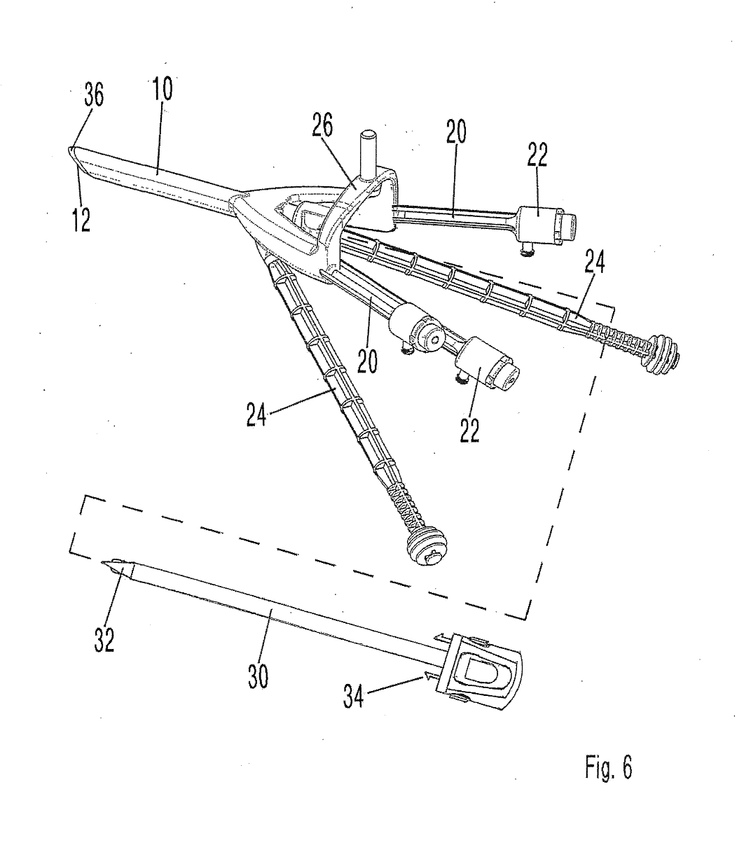

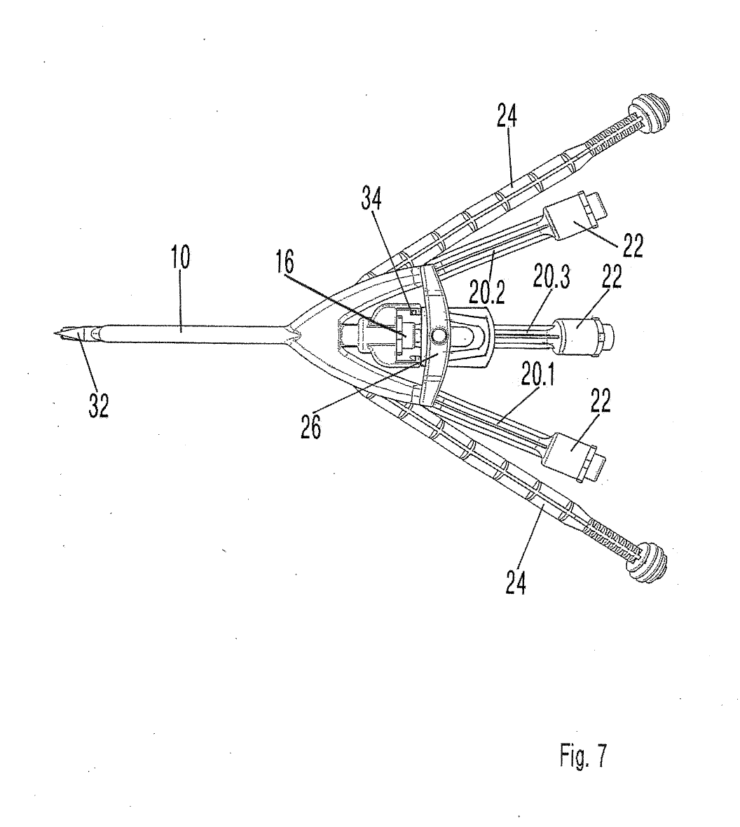

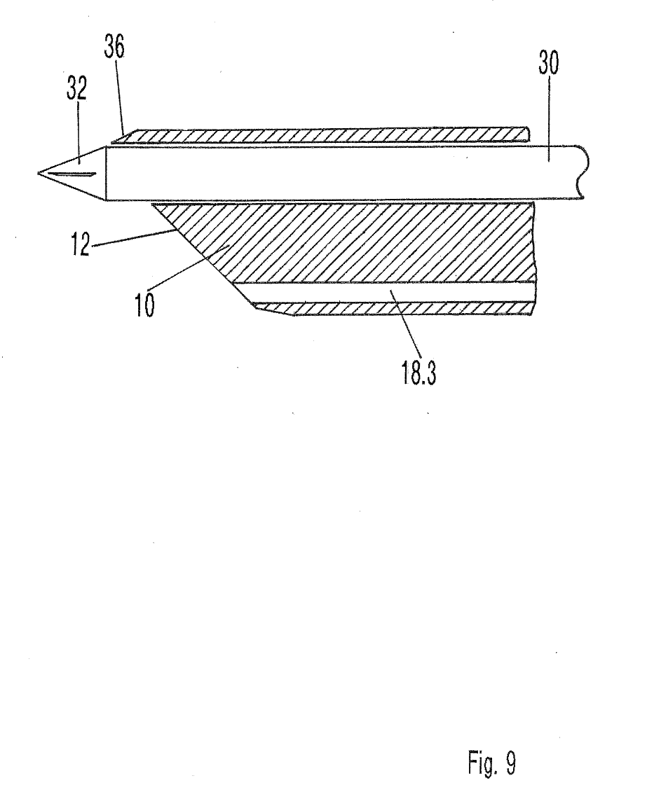

The instrument system also has an obturator 30. The obturator 30 is designed as a hollow cannula with a transparent distal tip 32. The tip 32 may be designed in a sharp or blunt conical shape. An endoscopic optical system is slid into the obturator 30. The optical system is designed in a manner known in and of itself and therefore is not shown in the drawing and will not be described in further detail. As is shown in FIGS. 6 and 7, the obturator 30 is slid through the valve block 16 into the inner channel 14 of the instrument shaft 10. With suitable fixing means 34 the obturator 30 introduced into the inner channel 14 is fixed onto the valve block, so that its axial position in the instrument shaft 10 is defined and maintained. The distal tip 32 of the obturator 30 in this position projects distally beyond the front face 12 of the instrument shaft 10, as FIG. 9 shows.

The outer diameter of the obturator 30 is adapted to the inner diameter of the inner channel 14 in such a way that the obturator 30 fits tightly in the inner channel 14 at the front face 12. This ensures that tissue cannot penetrate into the inner space of the interior channel 14. When the obturator 30 is inserted, the outer circumference of the projecting tip 32 merges continuously into the front face 12, as shown in FIG. 9. In the proximally located region (FIG. 9, bottom) this is apparent from the oblique position of the front face 12. In the distally located area (FIG. 9, top) the outer edge of the front face 12 between the outlet opening of the inner channel 14 and the circumference of the instrument shaft 10 is bordered by a bevel 36 in such a way that here also a continuous transition is achieved. The front face 12 thus merges across the entire circumference of the obturator tip 32, in a distally acute angle of 45° in the example shown, with the outer circumference of the obturator 30.

After removal of the obturator 30 from the instrument shaft 10, the endoscopic optical system can be withdrawn from the obturator 30 and can also be passed through the valve block 16 into the inner channel 14 of the instrument shaft 10 and fixed in the inner shaft 10 in its axial position. If the optical system alone is introduced into the instrument shaft 10 and especially fixed with a clamp 38 for the optical system, the distal end of the optical system lies in the plane of the front face 12 of the instrument shaft 10.

Semi-flexible instruments can be introduced to the working channels 18. Such semi-flexible instruments are known in and of themselves, so they need not be described in further detail here.

The semi-flexible instruments have a flexible, pliable shaft, stable against tensile and compression forces. The instruments can be introduced through the respective working channel 18, wherein because of the flexible shaft they can follow the curvature of the working channels 18. Once the instrument is introduced into the respective working channel 18, its distal working element projects distally out of the end face 12 of the instrument shaft 10. The proximal operating elements of the instrument remain proximally outside of the working channel 18 and its valve 22. The instrument is sealed off by the valve 22. Semi-flexible instruments used may be any of a plurality of instruments known in and of themselves with correspondingly designed working elements and operating elements. These can be, for example, cutting, clamping, grasping, or coagulating instruments or suction-irrigation instruments or biopsy forceps, etc.

In addition, in particular, pressurized gas can be insufflated through the working channels 18 or the inner channel 14. It is also possible to introduce liquids through the working channels and suction it off, for example to rinse the surgical field and suction off blood and tissue fluid.

A minimally invasive surgical procedure in the tissue of a patient is conducted in the following way using the instrument system according to the present disclosure:

First the obturator 30 is placed in the instrument shaft 10 and fixed in its axial position in the instrument shaft 10 in such a way that the distal tip 32 projects distally out of the front face 12 of the instrument shaft 10. The instrument shaft 10 is introduced into the tissue through a skin incision if necessary.

In this process the distal tip 32 of the obturator penetrates the tissue. The advancement of the distal end in the tissue can be observed using the optical system through the transparent tip 32 of the obturator 30. The tissue penetrated by the tip 32 further undergoes blunt dissection by the continuously following distal end of the instrument shaft 10. Since the obturator 30 at the distal end is tightly enclosed in the inner channel 14 and the distal end of the instrument shaft 10, during this penetration of the tissue, no tissue can intrude into the inner channel 14. The distal openings of the working channels 18 if necessary can be closed by a suitable stylet, so that also no tissue can penetrate into the working channels 18.

The instrument shaft 10 is guided under visualization via the transparent tip 32 of the obturator 30 through the tissue until the distal end of the instrument shaft 10 is positioned in the surgical target area. Delicate introduction and maneuvering of the instrument shaft 10 is made easier for the operator by means of the hand grips 24. Once the distal end of the instrument shaft 10 is positioned in the area of the surgery, the instrument shaft 10 is rotated around its longitudinal axis, which is ideally facilitated by grasping the instrument on the bracket 26. When the instrument shaft 10 is rotated, the distal end of the instrument shaft 10, because of its beveling and the eccentric arrangement of the obturator 30, creates an annular cavity in the tissue. Pressurized gas can now be insufflated into this initially produced cavity though one of the working channels 18 or the inner channel 14, so that the tissue is forced aside and the initially artificially created gap is dilated to form a cavity.

Into this artificial cavity now created in front of the distal end of the instrument shaft 10, the semi-flexible instruments can be introduced through the working channels 18 to perform surgical steps in the surgical area. For this purpose the obturator 30 is withdrawn from the instrument shaft 10 since further penetration of the tissue is no longer necessary. In place of the obturator 30 an endoscopic optical system is now introduced into the inner channel 14, positioned in this inner channel 14, and fixed with the clamp 38 for the optical system. The optical system previously disposed in the obturator 30 can preferably be used for this purpose. The optical system used and positioned allows a direct view into the artificial cavity created by the gas insufflation, so that the surgical steps can be performed under unimpeded direct view through the optical system. In this way a surgical site is produced, similar to that in a preformed body cavity during laparoscopic surgery.

The mutual arrangement of the appendages 20 and the hand grips 24 in this process allows ergonomically advantageous handling of the instrument system by the operator. The operator can grasp one of the hand grips 24 with one hand and hold it using the ring finger and the little finger, while simultaneously operating the actuation elements of an instrument in the adjacent appendage 20.1 or 20.2 with the same hand. By means of the bracket 26 and the connecting piece 28 the instrument system can also be held on a suitable stand to hold the instrument shaft 10 in its appropriate position during the surgical operation.

### LIST OF SYMBOLS

10 Instrument shaft
12 Front face
14 Inner channel
16 Valve block
18 Working channel
20 Appendage
22 Valve
24 Hand grip
26 Bracket
28 Connector
30 Obturator
32 Tip
34 Fixing means
36 Bevel
38 Optical system clamp

1. An instrument system, comprising:
an instrument shaft comprising:
a distal end insertable in to a patient body, wherein the distal end has a distal end face;
a proximal end that remains outside of the patient body;
a continuous inner channel passing axially through the instrument shaft;
a working channel passing axially and continuously in the instrument shaft;
an obturator that can coaxially receive an optical system, wherein the obturator is insertable in the continuous internal channel in such a manner that a transparent distal tip of the obturator projects out of the distal end face of the instrument shaft;
wherein a valve block is attached at the proximal end of the instrument shaft, the valve block closes off the inner channel and allows for sealed passage of the obturator or the optical system;
wherein distal end face of the instrument shaft is beveled at an angle different from 90° against a middle axis of the instrument shaft; and
wherein the inner channel and the working channel of the instrument shaft emerge in the distal end face and when the obturator is inserted, the transparent distal tip of the obturator merges continuously into the distal end face of the instrument shaft.

2. The instrument system according to claim 1, wherein the distal end face of the instrument shaft is beveled against the middle axis of the instrument shaft at an angle between 30° and 60°.

3. The instrument system according to claim 1, wherein the continuous inner channel emerges eccentrically relative to the middle axis of the instrument shaft in a distally located surface area of the distal end face.

4. The instrument system according to claim 1, wherein a distally located outer edge of the distal end face between an outlet opening of the inner channel and an outer circumference of the distal end face is angled with a bevel in such a manner that the distal end face in an area of the bevel merges at an acute angle into the outer circumference of the distal tip of the obturator projecting from the distal end face.

5. The instrument system according to claim 1, wherein the working channel, in a proximal end region, is angled against a longitudinal axis of the instrument shaft in a proximately opening angle merges into an appendage molded on at the proximal end of the instrument shaft and a hand grip is disposed at the proximal end of the instrument shaft.

6. The instrument system according to claim 5, wherein the working channel comprises three working channels disposed in the instrument shaft, the three working channels proceed axially parallel to the inner channel on a same side adjacent to the inner channel and emerge distally into a proximally located area of the distal end face;
wherein, in proximal end regions, each of the three working channels merge into appendages; and
wherein two appendages fall in a plane enclosing the longitudinal axis of the instrument shaft, while the third appendage proceeds at an angle to the plane enclosing the longitudinal axis of the instrument shaft.

7. The instrument system according to claim 5, further comprising two hand grips formed at the proximal end of the instrument shaft, each hand grip of the two hand grips having a rod shape and being angled at a proximally opening angle relative to the axis of the instrument shaft and against one another.

8. The instrument system according to claim 7, wherein each of the hand grips is disposed beneath one of the appendages, which is disposed in a plane with a middle axis of the instrument shaft.

9. The instrument system according to claim 5, wherein each of the appendages of the working channel has a valve which closes the working channel and allows for sealed passage of an instrument.

10. The instrument system according to claim 1, wherein the optical system incorporated in the obturator can be removed from the obturator and in place of the obturator, can be introduced through the valve block into the inner channel of the instrument shaft and can be fixed with a clamp for the optical system.

11. The instrument system according to claim 1, wherein a working channel or the inner channel may be used as an insufflation channel.

12. The instrument system according to claim 1, a retaining structure is disposed at the proximal end of the instrument shaft, allowing axially positioned fixation of the obturator.

13. The instrument system according to claim 1, wherein a clamp for the optical system is disposed at the proximal end of the instrument shaft.

14. The instrument system according to claim 1, further comprising a fastener molded on the instrument shaft that secures the instrument shaft to an operating stand.

15. An instrument, comprising:
an instrument shaft with a middle axis;
a distal end of the instrument shaft, the distal end insertable into a patient body and having a distal end face, the distal end face being beveled at a non-90° angel against the middle axis of the instrument shaft;
a proximal end of the instrument shaft, the proximate end remaining outside of the patient body when the distal end is inserted into the patient body;
an inner channel passing axially through the instrument shaft;
a valve block at the proximal end of the instrument shaft, the valve block closing off the inner channel and allows for sealed passage of an obturator with a distal transparent tip;
a working channel passing axially through the instrument shaft;
wherein the inner channel and the working channel are separate from one another;
wherein the working channel is configured to receive the obturator such that the transparent distal tip of the obturator projects out of the distal end face of the instrument shaft; and
wherein the inner channel and the working channel of the instrument shaft emerge in the distal end face and when the obturator is inserted, the transparent distal tip of the obturator merges continuously into the distal end face of the instrument shaft.

16. The instrument according to claim 15, wherein the distal end face of the instrument shaft is beveled against the middle axis of the instrument shaft at an angle between 30° and 60°.

17. The instrument according to claim 15, wherein the inner channel emerges eccentrically relative to the middle axis of the instrument shaft in a distally located surface area of the distal end face.

18. An instrument system, comprising:
an instrument, comprising:
an instrument shaft with a middle axis;
a distal end of the instrument shaft, the distal end insertable into a patient body and having a distal end face, the distal end face being beveled at a non-90° angel against the middle axis of the instrument shaft;
a proximal end of the instrument shaft, the proximate end remaining outside of the patient body when the distal end is inserted into the patient body;
an inner channel passing axially through the instrument shaft;
a valve block at the proximal end of the instrument shaft, the valve sealing the inner channel;
a plurality of working channels passing axially through the instrument shaft;
wherein the inner channel and the plurality of working channels are separate from one another;
an obturator with a distal tip;
wherein at least one working channel of the plurality of working channels is configured to receive the obturator such that the transparent distal tip of the obturator projects out of the distal end face of the instrument shaft; and
wherein the inner channel and the at least one working channel of the plurality of working channels emerge in the distal end face and when the obturator is inserted, the transparent distal tip of the obturator merges continuously into the distal end face of the instrument shaft.

19. The instrument system according to claim 18,
wherein the distal end face of the instrument shaft is beveled against the middle axis of the instrument shaft at an angle between 30° and 60°; and
wherein the continuous inner channel emerges eccentrically relative to the middle axis of the instrument shaft in a distally located surface area of the distal end face.

20. The instrument system according to claim 18, wherein the inner channel emerges eccentrically relative to the middle axis of the instrument shaft in a distally located surface area of the distal end face.
